(19) United States
(12) Patent Application Publication
(10) Pub. No.: US 2025/0229308 A1
(43) Pub. Date: Jul. 17, 2025

(54) TILTED KITCHEN WASTE DISPOSER

(71) Applicant: FOSHAN UDM APPLIANCES CO., LTD., Foshan (CN)

(72) Inventors: Weidong ZHANG, Foshan (CN); Wenbiao XIAO, Foshan (CN); Cuiling LIAO, Foshan (CN); Heng LOU, Foshan (CN)

(73) Assignee: FOSHAN UDM APPLIANCES CO., LTD., Foshan (CN)

(21) Appl. No.: 18/703,313

(22) PCT Filed: Oct. 11, 2022
(86) PCT No.: PCT/CN2022/124508

§ 371 Date: Apr. 19, 2024

(30) Foreign Application Priority Data
Jun. 14, 2022 (CN) 202221486962.9

(51) Int. Cl. B09B 3/35; A61L 9/014; B01D 46/00; B01D 53/04; B09B 3/38; B09B 101/70

(52) U.S. Cl. CPC B09B 3/35; A61L 9/014; B01D 46/0036; B01D 46/0038; B01D 53/04; B09B 3/38; A61L 2209/14; B01D 2253/102; B01D 2257/90; B09B 2101/70

(57) ABSTRACT

A tilted kitchen waste disposer, including a housing, a kitchen waste bin which is arranged on the housing, wherein the kitchen waste bin is provided with a stirring chamber. The housing is provided with an upper end opening that connects with the stirring chamber and an upper cover body which is arranged on the upper end opening, and on one side of the housing is provided with a supporting portion that incline from the side of the housing to the bottom of the housing. When the supporting portion contacts with the horizontal plane, the upper end face of the housing forms a certain angle with the horizontal plane. In the present invention, by setting the supporting portion that inclines from the side of the housing to the bottom of the housing, the upper end face of the housing forms a certain angle with the horizontal plane when the supporting portion contacts with the horizontal plane, which can facilitate users to observe the internal condition of the kitchen waste bin from the upper end face of the housing and put kitchen waste therein. Thus, the users do not have to observe the internal condition of the kitchen waste bin directly above the kitchen waste bin, which enhances experience of users.

### CROSS-REFERENCE TO RELATED APPLICATIONS

The application claims priority to Chinese patent application No. 202221486962.9, filed on Jun. 14, 2022, the entire contents of which are incorporated herein by reference.

### TECHNICAL FIELD

The present invention relates to the technical field of kitchen waste disposer, and particularly relates to a tilted kitchen waste disposer.

### BACKGROUND

Existing kitchen waste disposers are all in vertical structure. In order to improve its processing capability, it is necessary to increase large space of the stirring chamber, which results in a relatively large volume of kitchen waste bin. Before throwing kitchen waste, users need to be located directly above the kitchen waste bin to observe the condition of the kitchen waste bin and then put waste therein. When wanting to observe the process of kitchen waste, users have to open the cover and observe from directly above the kitchen waste bin. The inconvenient observation of the kitchen waste disposer makes a poor customer experience.

### SUMMARY

In response to the technical problems as mentioned above, such as the inconvenient observation of the kitchen waste disposer that makes poor customer experience, the technical solutions adopted by the present invention to solve the technical problems are:

A tilted kitchen waste disposer, comprising a housing, a kitchen waste bin which is arranged on the housing, wherein the kitchen waste bin is provided with a stirring chamber. The housing is further provided with an upper end opening that connects with the stirring chamber and an upper cover body which is arranged on the upper end opening, and on one side of the housing is provided with a supporting portion that inclines from the side of the housing to the bottom of the housing. When the supporting portion contacts with a horizontal plane, the upper end face of the housing forms a certain angle with the horizontal plane.

Further, the upper side of the housing is provided with a interactive assembly.

Further, the upper cover body is provided with a transparent window.

Further, the housing is provided with an air purification portion that connects with the stirring chamber. The air purification portion comprises an air intake filtration chamber, a power unit, an odor filtration chamber, and a purification exhaust port arranged in sequence. The air intake filtration chamber connects with the stirring chamber. The odor filtration chamber connects with the purification exhaust port. The power unit is arranged between the air intake filtration chamber and the odor filtration chamber. The purification exhaust port is arranged at the bottom of the housing.

Further, the odor filtration chamber is provided with an activated carbon filter element for filtering air.

Further, the air intake filtration chamber is provided with an air purification intake. The air purification intake is arranged on the inner side of the housing and locates between the upper end opening and the upper cover body.

Further, the kitchen waste bin is further provided with a heating assembly and a stirring assembly. The stirring assembly is arranged in the stirring chamber. The heating assembly is arranged at the bottom of the kitchen waste bin.

Further, the housing is provided with a driving portion that connects with the stirring assembly. The driving portion is used for driving the stirring assembly to rotate and stir in the stirring chamber.

Further, the stirring assembly is provided with a breaking portion and a stirring portion. The breaking portion is used for cutting and breaking kitchen waste, and the stirring portion is used for stirring kitchen waste.

The present invention has the following beneficial effects:

1. The present invention is provided with a supporting portion that inclines from the side of the housing to the bottom of the housing. When the supporting portion contacts with the horizontal plane, the upper end face of the housing forms a certain angle with the horizontal plane, which can facilitate the user to observe the internal condition of the kitchen waste bin from the upper end face of the housing and put kitchen waste therein. Thus, the users do not have to observe the internal condition of the kitchen waste bin directly above the kitchen waste bin, which enhances the experience of users.

2. In the present invention, when the stirring assembly is stirring kitchen waste, the stirring chamber forms a certain angle with the horizontal plane when the supporting portion contacts with the horizontal plane. As the stirring chamber is parallel with the bottom of the stirring chamber, the stirring portion turning kitchen waste upward continuously, so that the contact area with the air is increased, which can accelerate the speed of water evaporation and improve the processing efficiency.

3. In the present invention, by setting an air purification portion, the air generated in the process of processing kitchen waste can be disposed first and then be discharged, which reduces the odor of the processed kitchen waste, and at the same time accelerates the water evaporation in the kitchen waste bin, reduces the humidity in the kitchen waste bin, and greatly improves the processing efficiency.

The present invention will be further described below according to the accompanying drawings and specific embodiments.

### BRIEF DESCRIPTION OF DRAWINGS

FIG. 1 is a schematic view of a tilted kitchen waste disposer of the present invention.

FIG. 2 is a cutaway view of the tilted kitchen waste disposer of the present invention.

FIG. 3 is an exploded view of the tilted kitchen waste disposer of the present invention.

FIG. 4 is a schematic view of the tilted kitchen waste disposer of the present invention.

FIG. 5 is a schematic view of the tilted kitchen waste disposer of the present invention.

### DETAILED DESCRIPTION OF THE EMBODIMENTS

Embodiments of the present invention are described in detail below in association with the accompanying drawings.

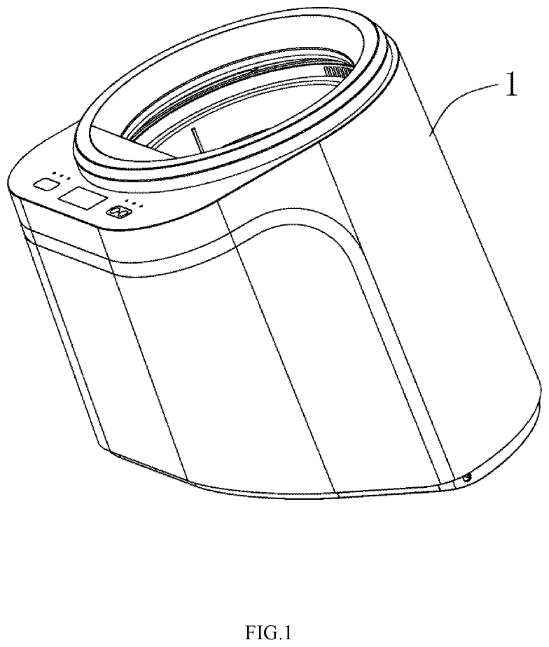

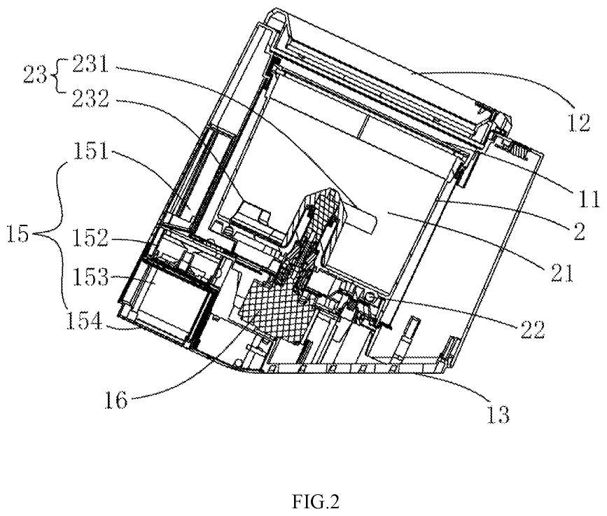

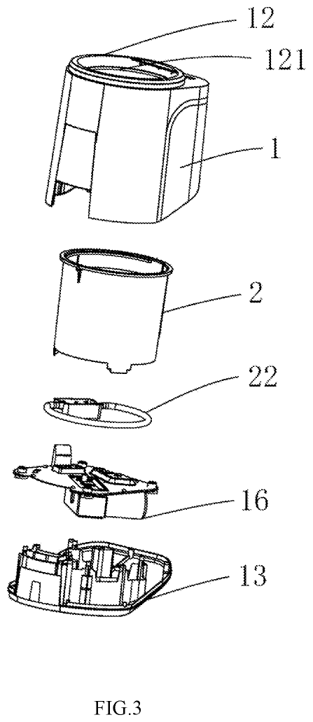

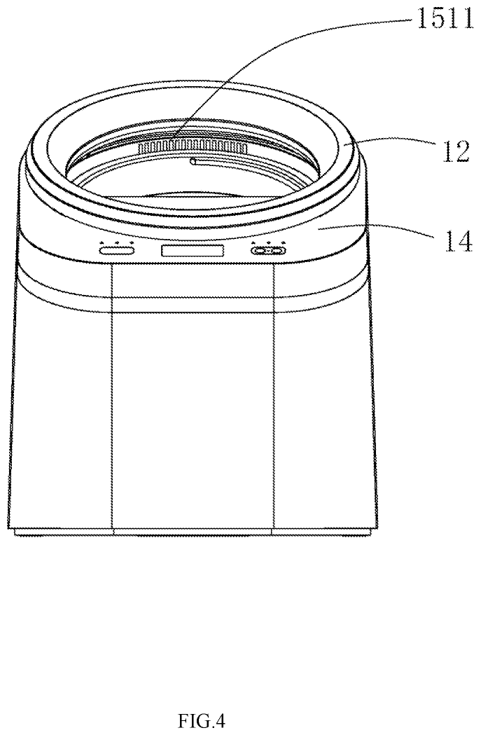

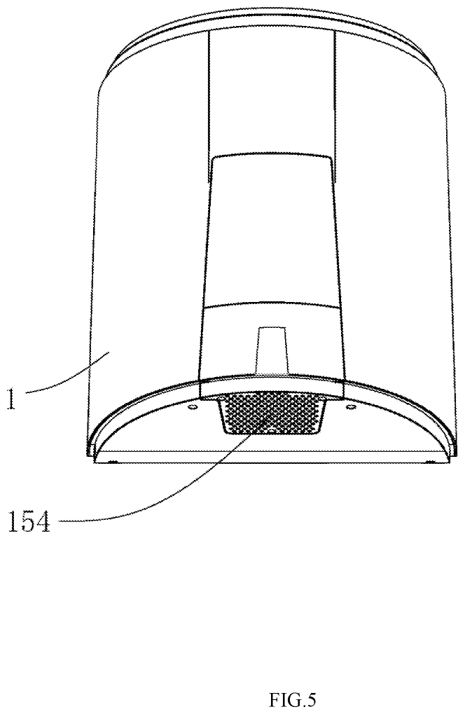

A tilted kitchen waste disposer as presented in FIG. 1, FIG. 2, FIG. 3, FIG. 4 and FIG. 5, comprising a housing 1, a kitchen waste bin 2 which is arranged in the housing 1, wherein the kitchen waste bin 2 is provided with a stirring chamber 21. The housing 1 is provided with an upper end opening 11 that connects with the stirring chamber 21 and an upper cover body 12 which is arranged on the upper end opening 11, and on one side of the housing is provided with a supporting portion 13 that incline from the side of the housing 1 to the bottom of the housing 1 on one side of the housing 1. When the supporting portion 13 contacts with the horizontal plane, the upper end face of the housing 1 forms a certain angle with the horizontal plane. In the present invention, by setting the supporting portion that inclines from the side of the housing to the bottom of the housing. When the supporting portion contacts with the horizontal plane, the upper end face of the housing forms a certain angle with the horizontal plane, which can facilitate the user to observe the internal condition of the kitchen waste bin from the upper end face of the housing and put kitchen waste therein. Thus, the users do not have to observe the internal condition of the kitchen waste bin directly above the kitchen waste bin, which enhances experience of the users.

The tilted kitchen waste disposer as presented in FIG. 4, wherein the upper side of the housing 1 is provided with an interactive assembly 14 and the upper cover body 12 that is provided with a transparent window 121. With such design, when it is necessary to observe the processing condition in the kitchen waste bin, there is no need to view directly above the kitchen waste bin to observe the internal condition of the kitchen waste bin. The transparent window can be used to observe the internal condition of the kitchen waste bin directly from the upper end face of the housing, and the kitchen waste disposer can be controlled by the interactive component on the top of the housing, which enables easier observation and more convenient operation and enhances the experience of users.

The tilted kitchen waste disposer as presented in FIG. 2, FIG. 4 and FIG. 5, wherein the housing 1 is provided with an air purification portion 15 that connects with the stirring chamber 21. The air purification portion 15 comprises an air intake filtration chamber 151, a power unit 152, an odor filtration chamber 153, and a purification exhaust port 154 arranged in sequence. The air intake filtration chamber 151 connects with the stirring chamber 21. The odor filtration chamber 153 connects with the purification exhaust port 154. The power unit 152 is arranged between the air intake filtration chamber 151 and the odor filtration chamber 153. The purification exhaust port 154 is arranged at the bottom of the housing 1. The odor filtration chamber 153 is provided with an activated carbon filter element for filtering air. The air intake filtration chamber 151 is provided with an air purification intake 1511. The air purification intake 1511 is arranged on the inner side of the housing 1 and locates between the upper end opening 11 and the upper cover body 12. With such design, negative pressure is generated when the power unit is activated, so that the air in the stirring chamber enters through pressure into the air purification intake which locates in the inner side of the housing and locates between the upper end opening and the upper cover body. The power unit will transport the air in the air intake filtration chamber to the odor filtration chamber. The air will be discharged out of the housing from the purification exhaust port after being filtered by the activated carbon filter element in the odor filtration chamber. By setting the air purification portion, the air generated in the process of processing kitchen waste can be disposed first and then be discharged, which reduces the odor of the processed kitchen waste, and at the same time accelerates the water evaporation in the kitchen waste bin, reduces the humidity in the kitchen waste bin, and greatly improves the process efficiency.

The tilted kitchen waste disposer as presented in FIG. 2 and FIG. 3, the kitchen waste bin 2 is provided with a heating assembly 22 and a stirring assembly 23. The stirring assembly 23 is arranged in the stirring chamber 21. The heating assembly 22 is arranged at the bottom of the kitchen waste bin 2. The housing 1 is provided with a driving portion 16 that connects with the stirring assembly 23. The driving portion 16 is used for driving the stirring assembly 23 to rotate and stir in the stirring chamber 21. With such design, the driving portion can start rotation and drive the stirring module to rotate and stir continuously. When the bottom of the kitchen waste bin is heated, the kitchen waste can contact with the stirring chamber to evaporate water.

The tilted kitchen waste disposer as presented in FIG. 2 and FIG. 3, the stirring assembly 23 is provided with a breaking portion 231 and a stirring portion 232. The breaking portion 231 is used for cutting and breaking kitchen waste, and the stirring portion 232 is used for stirring kitchen waste. With such design, when the kitchen waste is put into the stirring chamber and the supporting portion contacts with the horizontal plane, the breaking portion can cut and break the kitchen waste into small pieces. While the stirring chamber forms a certain angle with the horizontal plane, as the stirring portion is parallel with the bottom of the stirring chamber, the stirring portion turning kitchen waste upward continuously, so that the contact area with the air is increased, which can accelerate the speed of water evaporation and improve the processing efficiency. The water in the kitchen waste evaporates through the constant stirring by the stirring assembly, and the kitchen waste is converted into granular fertilizer.

The embodiment of the present invention is carried out as follows:

By setting the supporting portion 13 that from the side of the housing 1 to the inner side of the bottom of the housing 1, the upper end face of the housing 1 can form a certain angle with the horizontal plane when the supporting portion 13 contacts with the horizontal plane. By opening the upper cover body 12, users can observe the internal condition of the kitchen waste bin 2 from the upper end face of the housing 1 and put kitchen waste therein.

After setting processing time and temperature on the interactive assembly 14 which is on the upper side of the housing 1 and then starting a power supply. The driving portion 16 starts driving the stirring module 23 to rotate and stir. The breaking portion 231 in the stirring assembly 23 cuts and breaks the kitchen waste, so that the kitchen waste can be converted into small pieces. While the stirring chamber 21 forms a certain angle with the horizontal plane, as the stirring portion 232 is parallel with the bottom of the stirring chamber 21, the stirring portion 232 turning the kitchen waste upward continuously, so that the contact area with the air is increased. The heating assembly 22 heats the bottom of the kitchen waste bin 2 and cooperates with the stirring portion 232, which can accelerate the speed of water evaporation and improve the processing efficiency. The water in the kitchen waste evaporates through the constant stirring by the stirring assembly 23, and the kitchen waste is converted into granular fertilizer.

Negative pressure is generated when the power unit 152 is activated, so that the air in the stirring chamber 21 enters through pressure into the air purification intake 1511 which locates in the inner side of the housing 1 and locates between the upper end opening 11 and the upper cover body 12. The power unit 152 will transport the air in the air intake filtration chamber 151 to the odor filtration chamber 153. The air will be discharged out of the housing 1 from the purification exhaust port 154 after being filtered by the activated carbon filter element in the odor filtration chamber 153. By setting the air purification portion 15, the air generated in the process of processing kitchen waste can be disposed first and then be discharged, which reduces the odor of the processed kitchen waste, and at the same time accelerates the water evaporation in the kitchen waste bin 2, reduces the humidity in the kitchen waste bin 2, and greatly improves the process efficiency.

There is no need to view directly above the kitchen waste bin 2 to observe the internal condition of the kitchen waste bin 2 when it is necessary to observe the processing condition in the kitchen waste bin 2. The transparent window 121 can be used to observe the internal condition of the kitchen waste bin 2 directly from the upper end face of the housing 1, and the kitchen waste disposer can be controlled by the interactive component 14 on the top of the housing 1, which enables easier observation and more convenient operation and enhances the experience of users.

The foregoing is only an embodiment of the present invention to further explain the technical content of the invention, so as to make it easier for readers to understand, and is not intended to limit the present invention, and any modifications, equivalent substitutions and improvements made within the spirit and principles of the present invention shall be included in the scope of protection of the present invention. The scope of protection of the invention is subject to the claims.

What is claimed is:

1. A tilted kitchen waste disposer, comprising a housing, a kitchen waste bin which is arranged on the housing, wherein the kitchen waste bin is provided with a stirring chamber, the housing is further provided with an upper end opening that connects with the stirring chamber and an upper cover body which is arranged on the upper end opening, on one side of the housing is provided with a supporting portion that inclines from the side of the housing to a bottom of the housing, when the supporting portion contacts with a horizontal plane, an upper end face of the housing forms a certain angle with the horizontal plane.

2. The tilted kitchen waste disposer according to claim 1, wherein an upper side of the housing is provided with an interactive assembly.

3. The tilted kitchen waste disposer according to claim 1, wherein the upper cover body is provided with a transparent window.

4. The tilted kitchen waste disposer according to claim 1, wherein the housing is provided with an air purification portion that connects with the stirring chamber, the air purification portion comprises an air intake filtration chamber, a power unit, an odor filtration chamber, and a purification exhaust port arranged in sequence, the air intake filtration chamber connects with the stirring chamber, the odor filtration chamber connects with the purification exhaust port, the power unit is arranged between the air intake filtration chamber and the odor filtration chamber, the purification exhaust port is arranged at the bottom of the housing.

5. The tilted kitchen waste disposer according to claim 4, wherein the odor filtration chamber is provided with an activated carbon filter element for filtering air.

6. The tilted kitchen waste disposer according to claim 4, wherein the air intake filtration chamber is provided with an air purification intake, the air purification intake is arranged on an inner side of the housing and locates between the upper end opening and the upper cover body.

7. The tilted kitchen waste disposer according to claim 1, wherein the kitchen waste bin is further provided with a heating assembly and a stirring assembly, the stirring assembly is arranged in the stirring chamber, the heating assembly is arranged at the bottom of the kitchen waste bin.

8. The tilted kitchen waste disposer according to claim 7, wherein the housing is provided with a driving portion that connects with the stirring assembly, the driving portion is used for driving the stirring assembly to rotate and stir in the stirring chamber.

9. The tilted kitchen waste disposer according to claim 7, wherein the stirring assembly is provided with a breaking portion and a stirring portion, the breaking portion is used for cutting and breaking kitchen waste, and the stirring portion is used for stirring kitchen waste.
